(19) United States
(12) Patent Application Publication
(10) Pub. No.: US 2012/0082657 A1
(43) Pub. Date: Apr. 5, 2012

(54) SKIN PREPARATION CONTAINING FERMENTED SOYBEAN PASTE

(76) Inventor: Ji Soo YIM, Seoul (KR)

(21) Appl. No.: 12/897,335

(22) Filed: Oct. 4, 2010

(51) Int. Cl. A61K 35/74; A61P 17/00

(52) U.S. Cl. 424/115

(57) ABSTRACT

The present invention relates to a skin preparation containing fermented soybean paste, which has been traditionally called as ‘Chungkookjang’ in Korea. Further, the skin preparation of present invention has a useful effect for preventing or treating the atopic dermatitis by the active ingredients contained in Chungkookjang. Further, the skin preparation consists of soap and lotion containing fermented soybean paste, wherein i) said soap contains the high molecular weight materials or water insoluble materials in fermented soybean paste, such as, edible fiber, polysaccharide, proteins, lipids and/or water insoluble health functional materials; and ii) said lotion contains the low molecular weight materials or water soluble ions in fermented soybean paste, such as, low molecular weight of sugar, oligo-saccharide, amino acid, peptide and/or water soluble health functional materials

### BACKGROUND OF THE INVENTION

1. Field of the Invention

The present invention relates to a skin preparation containing fermented soybean paste, which has been traditionally called as ‘Chungkookjang’ in Korea. Further, the skin preparation of present invention has a useful effect for preventing or treating the atopic dermatitis by the active ingredients contained in Chungkookjang.

2. Description of Prior Art

Chungkookjang is one of Korean traditional fermented foods, which has been prepared by fermenting the soybean by the bacteria mainly consisting of Bacillus Subtilis. Chungkookjang seems to be similar to Japanese ‘Natto’ which has been world widely known as health functional fermented food. However, Chungkookjang has been known as better health functional fermented food by its unique ingredients, such as, various kinds of soybean proteins or peptides, edible fiber, phospholipids, isoflavon, saponin, phytic acid and/or phyto-estrogen. Further, Chungkookjang has lots of unidentified functional ingredients which have been obtained by the fermentation of soybean, which have been considered as health functional materials.

Recently, some scientists have revealed that some ingredients in Chungkookjang can prevent or treat the blood clot. Therefore, it can be useful for preventing or treating cardiovascular disease or hypertension.

In Korea, Chungkookjang has been sold in any traditional market. Recently, Chungkookjang can be prepared in home by convenient fermenting device. The steps for preparing Chungkookjang are as follows; i) boiling the washed soybean in the pot, ii) putting the boiled soybean with Bacillus Subtilis seed bacteria in the fermenting device and iii) incubating and fermenting the soybean mixture in the fermenting device for 3-4 days. The shape of Chungkookjang seems to be yellow soybean paste.

Korean Patent No.10-880661 ‘Chungkookjang soap and its preparation method’ discloses that the soap has been prepared containing Chungkookjang as active ingredient. Further, this Chungkookjang soap has been disclosed as useful for skin whitening and anti aging, which can be accomplished by the anti-oxidant ingredient in Chungkookjang, such as, isoflavon or saponin. Further, this Chungkookjang soap can be useful for preventing dark circle in the face by suppressing the melanin pigment.

However, the dried and sieved Chungkookjang powder has been used as a raw material of this Chungkookjang soap. Therefore, full ingredients in Chungkookjang cannot be used as a raw material of Chungkookjang soap, because Chungkookjang has 2 types of ingredients, one is water soluble liquid phase and another is solid phase.

In the liquid phase of Chungkookjang, the low molecular weight materials or water soluble ions can be included, such as, low molecular weight of sugar, oligo-saccharide, amino acid, peptide and/or water soluble health functional materials. In the solid phase of Chungkookjang, the high molecular weight materials or water insoluble materials can be included, such as, edible fiber, polysaccharide, proteins, lipids and/or water insoluble health functional materials.

The problem to be solved is that the liquid phase ingredient of Chungkookjang cannot be available in the form of soap. The present invention has been completed by carrying out the idea that liquid phase ingredient of Chungkookjang can be available in the form of lotion or colloidal solution, while solid phase ingredient of Chungkookjang can be available in the form of soap. Therefore, 2 kinds of skin preparations of present invention, such as, soap and lotion can afford all kinds of ingredients in Chungkookjang in a separate manner.

### SUMMARY OF THE INVENTION

The object of present invention is to provide a skin preparation consisting of soap and lotion containing fermented soybean paste, wherein i) said soap contains the high molecular weight materials or water insoluble materials in fermented soybean paste, such as, edible fiber, polysaccharide, proteins, lipids and/or water insoluble health functional materials; and ii) said lotion contains the low molecular weight materials or water soluble ions in fermented soybean paste, such as, low molecular weight of sugar, oligo-saccharide, amino acid, peptide and/or water soluble health functional materials

The said health functional materials are selected from isoflavon, saponin, phytic acid and/or phyto-estrogen.

Further, said skin preparation has a health function for preventing or treating atopic dermatitis.

Further, said fermented soybean paste has been prepared by the steps comprising i) boiling the washed soybean in the pot, ii) putting the boiled soybean with Bacillus Subtilis seed bacteria in the fermenting device and iii) incubating and fermenting the soybean mixture in the fermenting device for 3˜4 days.

### BRIEF DESCRIPTION OF THE DRAWINGS

FIG. 1a and FIG. 1b show a soap containing fermented soybean paste of present application.

FIG. 2a shows an atopic dermatitis skin condition (arm) before treating the arm with soap and lotion of present application.

FIG. 2b shows an atopic dermatitis skin condition (arm) after treating the arm with soap and lotion of present application.

FIG. 3a shows an atopic dermatitis skin condition (foot) before treating the foot with soap and lotion of present application.

FIG. 3b shows an atopic dermatitis skin condition (foot) after treating the foot with soap and lotion of present application.

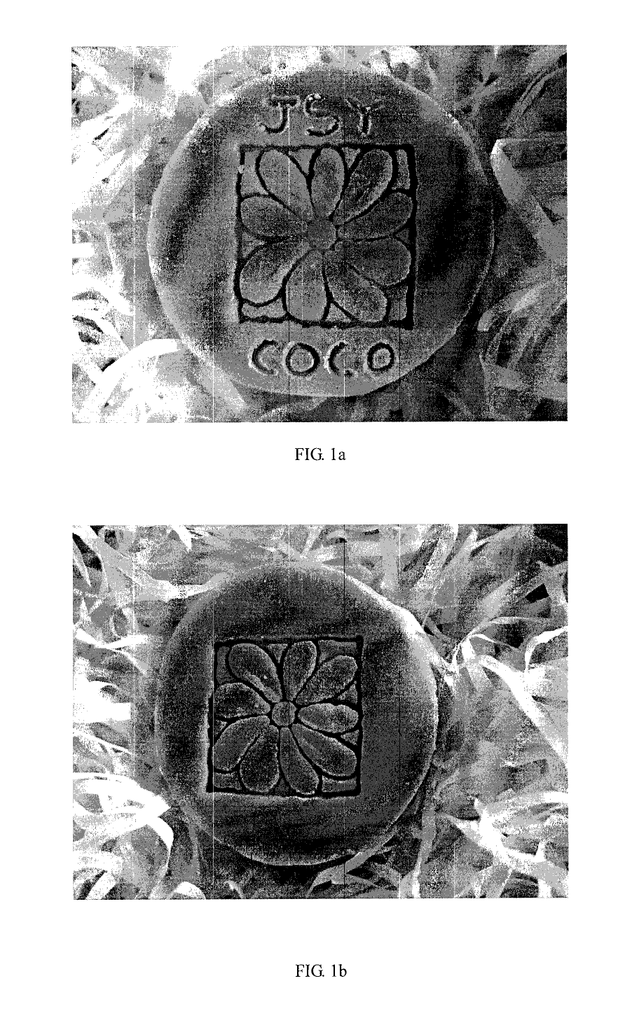

### DETAILED DESCRIPTION OF THE INVENTION

Present invention relates to a skin preparation consisting of soap and lotion containing fermented soybean paste, wherein i) said soap contains the high molecular weight materials or water insoluble materials in fermented soybean paste, such as, edible fiber, polysaccharide, proteins, lipids and/or water insoluble health functional materials; and ii) said lotion contains the low molecular weight materials or water soluble ions in fermented soybean paste, such as, low molecular weight of sugar, oligo-saccharide, amino acid, peptide and/or water soluble health functional materials.

Said fermented soybean paste is the same material of Korean traditional food ‘Chungkookjang’, which is prepared by fermenting the soybean by the bacteria mainly consisting of Bacillus Subtilis.

All kinds of soybean can be used as raw material of Chungkookjang. The preparation method for Chungkookjang can be anyone of traditional method or home made method. The home made Chungkookjang can be prepared by i) boiling the washed soybean in the pot, ii) putting the boiled soybean with Bacillus Subtilis seed bacteria in the fermenting device and iii) incubating and fermenting the soybean mixture in the fermenting device for 3˜4 days.

The soap containing fermented soybean paste can be made by any known method, as well as the lotion containing fermented soybean paste also can be made by any known method.

The atopic dermatitis is a kind of skin disease or syndrome caused by abnormal immune response against exogenic antigen, which is common to the baby or child in Korea. However, the treating method has not been developed. Only steroid hormone can relieve the symptoms of atopic dermatitis.

Therefore, many kinds of Korean or Chinese traditional herbs have been applied to relieve the symptom of atopic dermatitis. For example, the dried powder of Houttuynia Cordata has been known to be effective for relieving the symptom of atopic dermatitis. However, any Korean or Chinese traditional herb has not been dramatically effective to relieve the symptom of atopic dermatitis.

However, the inventor of present application has found that skin preparation containing fermented soybean paste is dramatically effective for preventing or treating atopic dermatitis.

The present application can be explained more specifically by following Examples. However, the scope of present invention cannot be limited to following Examples.

### EXAMPLES

### Preparation Example 1

### Preparation of Fermented Soybean Paste

Preparation of fermented soybean paste ‘Chungkookjang’ has been prepared by following steps. i) boiling 100 wt part of washed soybean in the pot, ii) putting the 100 wt part of boiled soybean, 10 wt part of water and 0.1 wt part of Bacillus Subtilis seed bacteria in the fermenting device and iii) incubating and fermenting the said soybean mixture in the fermenting device at 40° C. for 3˜4 days.

The obtained 100 wt part of fermented soybean paste has been added to 500 wt part of water. Then, the water soluble ingredients of Chungkookjang has been dissolved in water and water insoluble ingredient of Chungkookjang' has been remained as solid residue. Then, the supernatant has been filtered and concentrated in the reduced pressure at room temperature. Further, the remained solid residue has been dried at room temperature.

Finally, 150 wt part of concentrated solution and 95 wt part of solid residue have been obtained. The dried solid residue has passed through the sieves. Finally, the fine dried powder having less than 500 micrometer of particle size has been obtained from solid residue.

The obtained concentrated solution having water soluble ingredient of Chungkookjang has been used as an active ingredient of lotion. On the other hand, the obtained fine dried powder of Chungkookjang has been used as an active ingredient of soap.

### Preparation Example 2

### Preparation of Soap Having Dried Powder of Chungkookjang

100 wt part of soap base and 5 wt part of dried powder of Chungkookjang have been mixed at 70° C. by heating the mixture. Then, the mixture has been cooled at room temperature. Finally, the soap having dried powder of Chungkookjang has been obtained.

### Preparation Example 3

### Preparation of Soap Having Dried Powder of Chungkookjang

100 wt part of olive oil or palm oil, 13 wt part of sodium hydroxide, 37 wt part of water and 5 wt part of dried powder of Chungkookjang have been mixed at 40° C. Then, the mixture has been cooled at room temperature. Finally, the soap having dried powder of Chungkookjang has been obtained.

### Preparation Example 4

### Preparation of Soap Having Dried Powder of Chungkookjang and Houttuynia Cordata

100 wt part of soap base, 5 wt part of dried powder of Chungkookjang and 1 wt part of dried powder of Houttuynia Cordata have been mixed at 70° C. by heating the mixture. Then, the mixture has been cooled at room temperature. Finally, the soap having dried powder of Chungkookjang and Houttuynia Cordata has been obtained.

### Preparation Example 5

### Preparation of Lotion Having Concentrated Solution of Chungkookjang

100 wt part of lotion base (containing 95 wt part of water and 5 wt part of mixture of PEG, olive oil, emulsifier and ethanol) and 20 wt part of concentrated solution having water soluble ingredient of Chungkookjang have been mixed at 40° C. Then, obtained mixed lotion solution has been cooled at room temperature. Finally, the lotion having concentrated solution having water soluble ingredient of Chungkookjang has been obtained.

### Preparation Example 6

### Preparation of Lotion Having Concentrated Solution of Chungkookjang and Houttuynia Cordata

100 wt part of lotion base (containing 95 wt part of water and 5 wt part of mixture of PEG, olive oil, emulsifier and ethanol), 20 wt part of concentrated solution having water soluble ingredient of Chungkookjang and 1 wt part of dried powder of Houttuynia Cordata have been mixed at 40° C. Then, obtained mixed lotion solution has been cooled at room temperature. Finally, the lotion having concentrated solution having water soluble ingredient of Chungkookjang and Houttuynia Cordata has been obtained.

### Example

### Test For Health Function for Treating Atopic Dermatitis

1) Control Group: only application of soap prepared by Preparation Example 2

2) Experimental Group 1: application both soap prepared by Preparation Example 2 and lotion prepared by Preparation Example 5

3) Experimental Group 2: application both soap prepared by Preparation Example 4 and lotion prepared by Preparation Example 6

9 children having similar atopic dermatitis syndrome have been divided into 3 groups; Control Group, Experimental Group 1 and Experimental Group 2 respectively having 3 children in each group.

Control group has been applied into only soap prepared by Preparation Example 2 and applied 3 times a day for 2 months. Experimental Group 1 has been applied into soap prepared by Preparation Example 2 and lotion prepared by Preparation Example 5, and applied 3 times a day for 2 months. Experimental Group 2 has been applied into soap prepared by Preparation Example 4 and lotion prepared by Preparation Example 6, and applied 3 times a day for 2 months.

After 2 months application, we have measured how much atopic dermatitis syndrome has been relieved.

The Control Group showed a little relief of atopic dermatitis syndrome. More specifically, 2 children have been much relieved while 1 child has not been relieved. Both Experimental Group 1 and Experimental Group 2 showed better relief of atopic dermatitis syndrome. All 3 children have been much relieved.

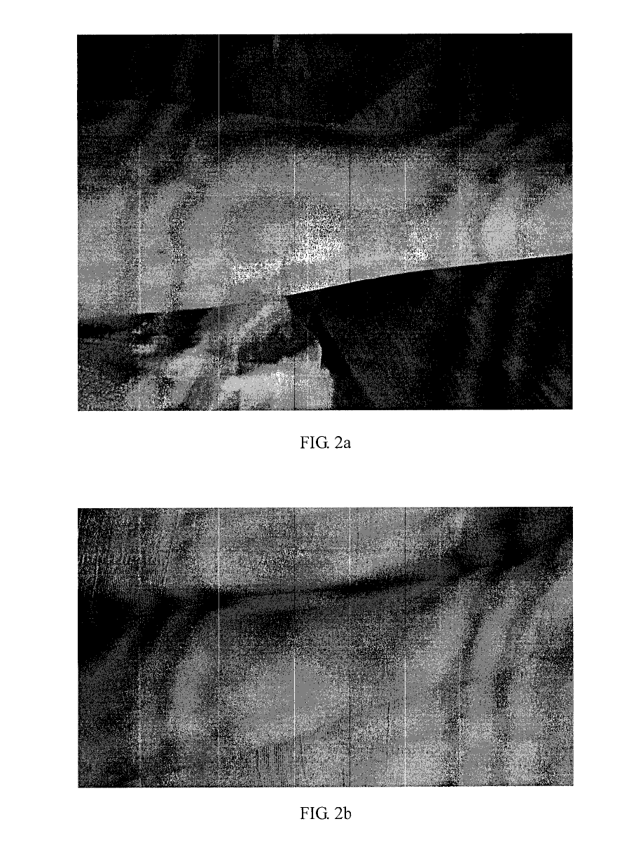

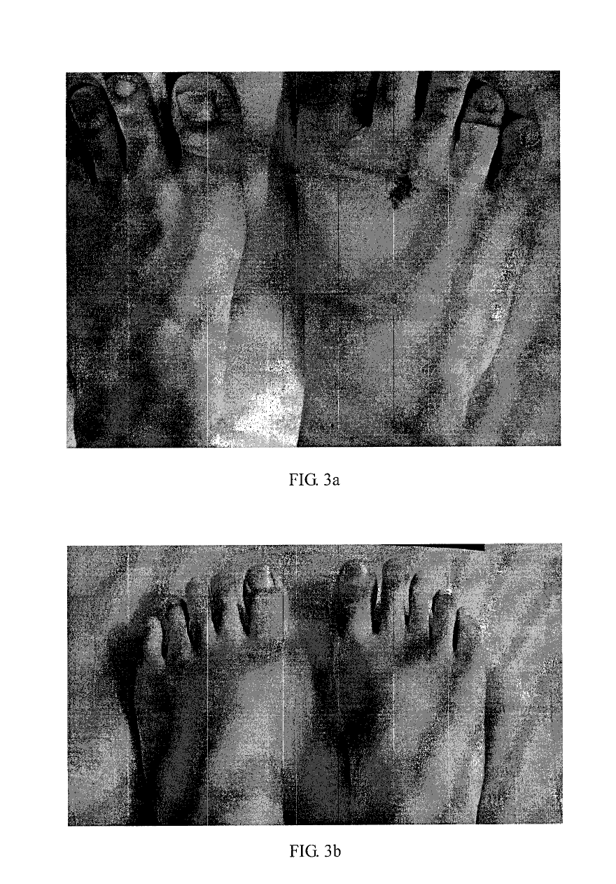

FIG. 2a shows an atopic dermatitis skin condition (arm) before treating the arm with soap and lotion to the subject in Experimental Group 1. FIG. 2b shows an atopic dermatitis skin condition (arm) after treating the arm with soap and lotion to the subject in Experimental Group 1. FIG. 3a shows an atopic dermatitis skin condition (foot) before treating the foot with soap and lotion to the subject in Experimental Group 1. FIG. 3b shows an atopic dermatitis skin condition (foot) after treating the foot with soap and lotion to the subject in Experimental Group 1.

As a conclusion, the both application of soap and lotion having ingredients of Chungkookjang can relieve the atopic dermatitis syndrome. In other words, the active ingredient for relieving atopic dermatitis syndrome may be contained in lotion form.

What is claimed is:

1. A skin preparation consisting of soap and lotion containing fermented soybean paste, wherein i) said soap contains the high molecular weight materials or water insoluble materials in fermented soybean paste, such as, edible fiber, polysaccharide, proteins, lipids and/or water insoluble health functional materials; and ii) said lotion contains the low molecular weight materials or water soluble ions in fermented soybean paste, such as, low molecular weight of sugar, oligo-saccharide, amino acid, peptide and/or water soluble health functional materials

2. The skin preparation according to claim 1, wherein said health functional materials are selected from isoflavon, saponin, phytic acid and/or phyto-estrogen.

3. The skin preparation according to claim 1, wherein said skin preparation has a health function for preventing or treating atopic dermatitis.

4. The skin preparation according to claim 1, wherein said fermented soybean paste has been prepared by the steps comprising i) boiling the washed soybean in the pot, ii) putting the boiled soybean with Bacillus Subtilis seed bacteria in the fermenting device and iii) incubating and fermenting the soybean mixture in the fermenting device for 3˜4 days.
